(19) United States
(12) Patent Application Publication
(10) Pub. No.: US 2004/0111015 A1
(43) Pub. Date: Jun. 10, 2004

(54) Patient monitor

(76) Inventor: Leland L. Ladd, Dunedin, FL (US)

Correspondence Address: James E. Bartek, 3830 - 56th Street, Grandville, MI, 49418, US

(21) Appl. No.: 10/725,749

(22) Filed: Dec. 2, 2003

Related U.S. Application Data
(63) Continuation-in-part of application No. 09/677,365, filed on Oct. 2, 2000, now Pat. No. 6,679,840.
(63) Continuation of application No. 09/834,548, filed on Apr. 13, 2001, now Pat. No. 6,656,149.
(60) Provisional application No. 60/156,856, filed on Sep. 30, 1999.
(60) Provisional application No. 60/196,946, filed on Apr. 13, 2000.

(51) Int. Cl. A61B 5/00

(52) U.S. Cl. 600/300

(57) ABSTRACT

A patient monitor has an information processor, an information display, a control or selector. The information processor is programmable and re-programmable according to a user's requirements. The sockets are substantially identical with a number of socket connectors and are electrically connected in parallel with one another. The sensor sockets and the sensors have connectors that connect when the sensors are plugged into the sockets. Not all of the connectors are used by a sensor, however, so the patient monitor identifies and differentiates each sensor not by which socket it is plugged into, but by which plug and socket connectors are used. In other aspects, the invention includes an expansible medical suction canister having an expansible chamber and a support. The chamber is expandable to an open position and compressible to a closed position. The chamber also has opposing first and second ends. The first and the second ends are spaced relatively farther apart in the open position than in the closed position. The support is connected between the opposing ends and moves with the opposing ends between the open and closed positions. The support also has a locked condition in the open position. The support may have a first element and a second element. The first and the second elements preferably move between open and closed positions with the opposing chamber ends. In one aspect, the support may be a telescoping member with the first element including a detent and the detent engaging the second element. Further, the detent may be biased toward engagement with the second element.

### CROSS-REFERENCES TO RELATED APPLICATIONS

This is a continuing application of co-pending U.S. patent application Ser. No. 09/677,365, entitled Patient Monitor and filed on 02 Oct. 2000 by Leland L. Ladd, now U.S. Pat. No. ______ and issued also ______, the disclosure of which is incorporated here by reference; which is a continuing application of co-pending U.S. patent application Ser. No. 60/156,856, entitled Patient Monitor and filed on 30 Sep. 1999 by Leland L. Ladd, now abandoned, the disclosure of which is incorporated here by reference. This is also a continuing application of co-pending U.S. patent application Ser. No. 09/834,548, entitled Expansible Medical Suction Canister and filed on 13 Apr. 2001 by Leland L. Ladd, the disclosure of which is incorporated here by reference, now U.S. Pat. No. 6,656,149 and issued 02 Dec. 2003; which is a continuing application of co-pending U.S. patent application Ser. No. 60/196,946, entitled Expansible Medical Suction Canister and filed on 13 Apr. 2000 by Leland L. Ladd and Gary A. Wagner, now abandoned, the disclosure of which is incorporated here by reference. This is also a continuing application of co-pending U.S. patent application Ser. No. ______, entitled Medical Bed Pad and filed on 12 Sep. 2003 by Kenneth Assink, the disclosure of which is incorporated here by reference, now co-pending.

### STATEMENT REGARDING FEDERALLY SPONSORED RESEARCH OR DEVELOPMENT

Not Applicable.

### REFERENCE TO A SEQUENCE LISTING

Not Applicable.

### BACKGROUND OF THE INVENTION

The invention relates generally to patient care and more particularly to the monitoring of a patient's condition and the issue of an alarm when predetermined criteria are met. A variety of personal condition indicators may require monitoring in various patient care settings, including home care, nursing home, and hospital environments.

It has become a self evident truth that competition reaches into virtually every segment of not only our national economy, but the world economy as well. The experts may discuss and argue without ending and without conclusion as to whatever may be the cause or reason of why the competitiveness of the economy is present. To some extent that is immaterial because it is here and must be accommodated or otherwise dealt with. At least one influence of the present competitiveness is an increasing demand on the available funds of anyone in the market place. This includes non-discretionary as well as discretionary purchase decisions.

More specific to the invention, a focus has been drawn to the medical field and the expense of medical care. The medical services industry is an enormous industry and continues to grow dramatically. The resources in terms of both people and funds, that are committed to providing medical care and services is incomprehensible to many. One effect of competitiveness in the economy, upon medical care is pressure toward efficiency, which may be key phrased as doing more with less. Medical care is transitioning, if it has not already transitioned, to management and administration by non-medically trained professional administrators. This is no longer a profession that is driven by the professional medical care givers.

Thus, consistent quality monitoring may too frequently be compromised in each setting, however. In a home setting, trained personnel are commonly not available or affordable, for example, and care can easily be overlooked. Similarly, trained nursing care personnel are commonly limited in a nursing home setting. This limitation may result from limited funding or cost reduction pressures. This limitation may also result from unusual or unforeseen circumstances in which more patients require attention from trained personnel at a given time than was expected or forecasted. That is to say, merely the inherent unpredictability of nursing care requirements may result in a personnel short fall.

Even in a hospital setting, personnel resources may fall short for some, if not all, of the same reasons as noted above. Further, the demands of an operating theatre can cause a lapse in patient monitoring, especially when combined with economic pressures. To say the very least, any effective assistance that may facilitate or enhance the work of an operating room nurse is clearly desirable. Even the smallest detail can make a life or death difference in surgery, including not requiring any time to identify which of several sockets on a piece of equipment is the correct socket, for example. Another of a multitude of aspects regarding efficiency in medical care is the task of eliminating medical waste in physical facilities.

Further, the professional managers and administrators continually identify and evaluate every factor and detail, large and small. Materials and logistics control is important in medical services as it is in any industry. One exemplary detail that adds to a big allocation of resources is in the area of inventory, including the inventory of medical supplies and the disposal of consumed supplies and other medical waste, which is a “bio-hazard” waste disposal concern.

It is also self evident, then, that reducing the allocation of resources to the storage and handling of medical supplies and to the disposal of medical waste is important. After all, the more resources that can be freed from the “back room” tasks to the “front office” dispensing of medical care, the better. More specifically as to fluids canisters, the typical practice is to inventory a supply of canisters in a number of sizes, say one, two, and three liter capacities, for example. Thus, the appropriate size canister may be selected for a given procedure. The physical storage space required for a proper supply of suction canisters may be reduced, freeing up allocated resources, if only one size canister is inventoried.

The problem here, then, is that a reduction in the variety of sizes that are inventoried may result in forcing the use of a larger than needed canister and associated increased disposal resources. A worse result is that a use of a number of small canisters may be imposed upon a medical procedure. Any medical procedure carries with it an inherent elevated stress or tension environment. Virtually any medical procedure also requires focused attention to various tasks. Thus, imposing an added requirement, and risk, of watching and changing small suction canisters is not justifiable.

Even the few, rudimentary factors noted above that are involved in the supply, use, and disposal of suction canisters demonstrate a desire for a one size canister that dose not compromise the dispensing of care or further burden disposal of “bio-hazard” waste. More particularly, these factors point to a desire for a suction canister that is small in storage of a supply inventory, provides maximum capacity during a medical procedure, and yet does not further burden waste disposal. So far, an expandable and collapsible suction canister has not been practical if only because a canister that will not collapse under suction during use in a medical procedure also commonly resists compression for disposal.

### BRIEF SUMMARY OF THE INVENTION

Accordingly, a patient monitor of the invention has an information processor, an information display, a control or selector, at least two sensor sockets, and at least two patient sensors. The sockets are substantially identical with a number of socket connectors and are electrically connected in parallel with one another. One of the sensors monitors one aspect of a patient's condition and generates a signal to the information processor accordingly. A second sensor monitors a second aspect of a patient's condition and generates a signal to the information processor accordingly. Each of the patient sensors plugs into any of the sensor sockets. The sensor sockets and the sensors have connectors that connect when the sensors are plugged into the sockets. Not all of the connectors are used by a sensor, however, so the patient monitor identifies and differentiates each sensor not by which socket it is plugged into, but by which plug and socket connectors are used.

In another aspect of the invention, an expansible medical suction canister of the invention has an expansible chamber and a support. The chamber is expandable to an open position and compressible to a closed position. The chamber also has opposing first and second ends. The first and the second ends are spaced relatively farther apart in the open position than in the closed position. The support is connected between the opposing ends and moves with the opposing ends between the open and closed positions. The support also has a locked condition in the open position. The support may have a first element and a second element. The first and the second elements preferably move between open and closed positions with the opposing chamber ends.

In one aspect, the first element may have a stop surface and the second element may include a leg with an end that abuts the stop surface in the open position, whereby compression of the canister to the closed position is resisted. In another aspect, the support may be a telescoping member with the first element including a detent and the detent engaging the second element. Further, the detent may be biased toward engagement with the second element.

These and other features, objects, and benefits of the invention will be recognized by one having ordinary skill in the art and by those who practice the invention, from this disclosure, comprising the specification, the claims, and the drawing figures.

### BRIEF DESCRIPTION OF THE SEVERAL VIEWS OF THE DRAWING

FIG. 1 is a front perspective view of a cabinet of a patient monitor according to the invention;

FIG. 2 is an exploded view thereof;

FIG. 3 is a projection of the front panel thereof, showing a preferred user interface;

FIG. 4 is a block diagram of a power circuit for the patient monitor;

FIG. 5 is a pump control diagram for the patient monitor;

FIG. 6 is a block diagram of a circuit therefor;

FIG. 7 is the view of FIG. 6 showing an alternative circuit;

FIG. 8 is a block diagram of a processor circuit for the patient monitor;

FIG. 9 is a software flow diagram for the patient monitor;

FIG. 10 is a schematic view of a mass level sensor for the patient monitor;

FIG. 11 is a schematic view of a fluid level sensor for the patient monitor;

FIG. 12 is a schematic view of a patient movement pad for the patient monitor; and

FIG. 13 is a schematic view of an array of connectors and input devices for the patient monitor.

FIG. 14 is a side elevational schematic view of an expansible medical suction canister according to the invention, shown in a compressed condition;

FIG. 15 is the view of FIG. 14, shown in an expanded condition;

FIG. 16 is a centerline cross-sectional view thereof, showing a first alternative embodiment with a first locking support member;

FIG. 17 is an enlarged fragmentary cross-sectional view of detail XVII-XVII of FIG. 16;

FIG. 18 is the view of FIG. 14, showing a first alternative;

FIG. 19 is the view of FIG. 14, showing a second alternative; and

FIG. 20 is a cross-sectional view along line XX-XX of FIG. 19; and

FIG. 21a is a perspective view of a first embodiment of a medical bed pad according to the present invention;

FIG. 22 is a side view of the medical bed pad in FIG. 21;

FIG. 23 is a side view of a second embodiment of a medical bed pad according to the present invention;

FIG. 24 is a perspective view of a third embodiment of a medical bed pad according to the present invention;

FIG. 25 is a perspective view of a fourth embodiment of a medical bed pad according to the present invention;

FIG. 26 is a perspective view of a fifth embodiment of a medical bed pad; and

FIG. 27 is a perspective view of a sixth embodiment of a medical bed pad in accordance with the present concept.

### DETAILED DESCRIPTION OF THE INVENTION

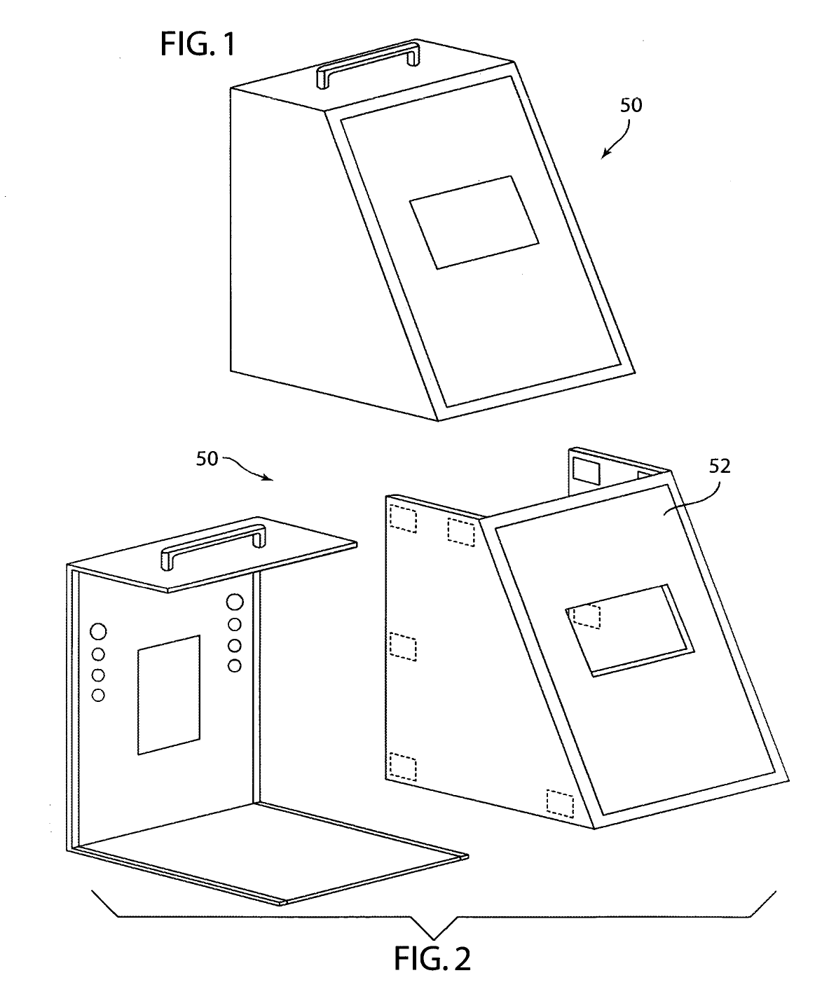

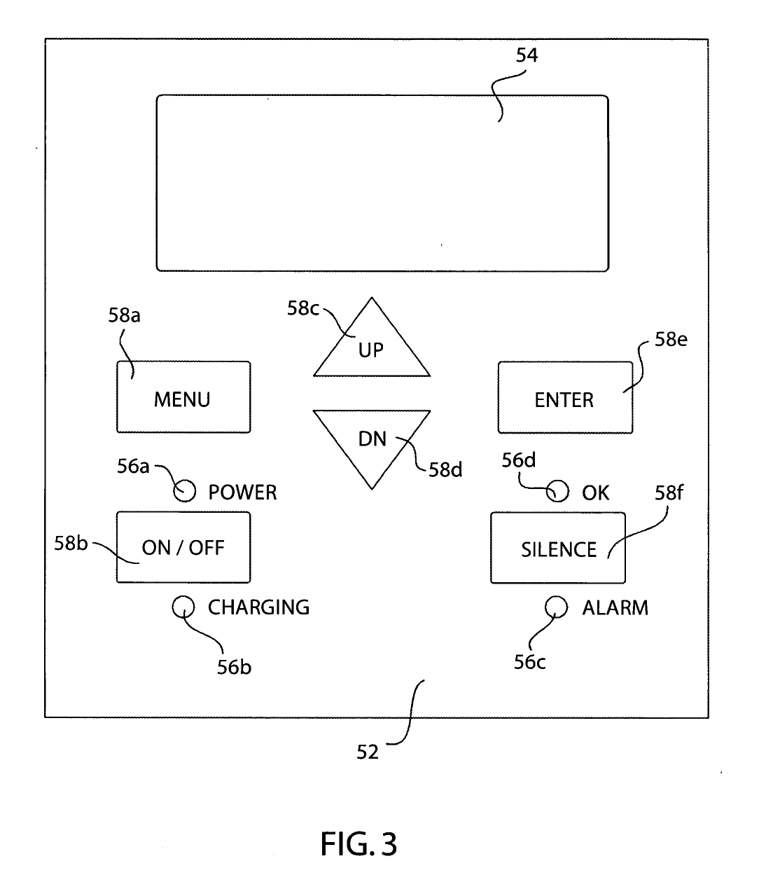

A preferred embodiment of a patient monitor according to the invention is generally shown in the drawing figures and discussed below. The patient monitor may be generally housed in a cabinet 50 as shown in drawing FIGS. 1-3. The cabinet may have a front panel 52 with a display panel 54, status indicators 56a-56d, and user input switches 58a-58f. The cabinet 50 may be of various constructions and configurations, as will be understood by one having ordinary skill in the art. Some exemplary cabinet materials may include, without limitation, stainless steel, powder coated metals, and engineering plastics.

The display panel 54 is preferably a liquid crystal display (LCD) although other display devices may be used depending upon a manufacturer's or user's preferences. More particularly, the inventor has found a 16×2 character LCD to perform well in the patient monitor. Additionally, a touch screen display may be used. A touch screen display may further incorporate various switching, selection, or input tasks, for example.

Likewise, light emitting diodes have been found to perform well for the various status indicators 56a-56d, although other indicators may be substituted. Presently, indicators for monitor power status (on/off) 56a, for batter power source charging status 56b, and for an alarm condition 56c and 56d relative to a patient monitor have been found to be sufficient and minimally required indicators. Additional indicators may be added and desired by various users, although potential users are cautioned against the inherent confusion that comes with a plethora of indicators.

A sufficient and minimal array of input switches is also shown in the drawings and includes switches for menu access 58a, power 58b, menu scrolling 58c and 58d, menu choice entry 58e, and alarm toggle 58f. Membrane tactile switching or the like, including without limitation devices that combine display screen and input as suggested above, may be preferred. Other switching may also be used with compromises in the ability to clean the front panel around the switches, however. While most of the switches are most preferably accessible on the front panel, the power switch 58b may alternatively be located on the back panel, for example.

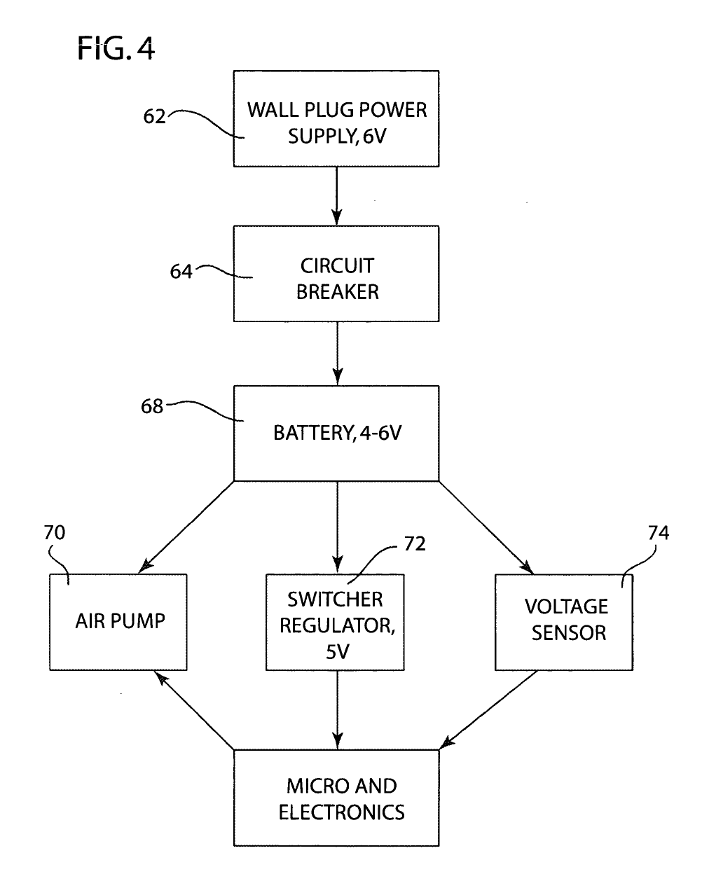

The monitor is preferably provided with an alternative choice between wall plug, also commonly known as line power, and an onboard battery power supply (FIG. 4). A power cord with an integral transformer and plug may be used as is commonly known to provide appropriate battery current to the patient monitor, which will be understood by one having ordinary skill in the art. By providing onboard battery power, the patient monitor may operate a nominal twenty-five hours, for example, during a power failure or patient transportation.

A circuit for the ability to choose alternative power sources is schematically shown in FIG. 4. The transformer power cord 62 feeds through a circuit breaker 64 to the battery 66. The battery 68 then feeds to an air pump 70, a power regulator 72, and a voltage sensor 74. The power regulator may be a switching type regulator of about five volts output, for example. The output of the regulator is then used for the electronics 80 of the patient monitor. A loop from the battery 68 to the voltage sensor 74 provides monitoring of the power supply and may send a low power signal to the control electronics 80 when battery power drops below a preselected value, as will be understood by one having ordinary skill in the art. This may occur after an extended period of being disconnected from wall power. A low power signal alarm may be interpreted as a flashing power indicator 56a or an audible voice alert. The low power alarm will preferably activate at power levels of about twenty-five percent, at fifteen percent, and again at five percent remaining charge. These power levels may be reflected in the power indicator 56a as increasing flashing frequency. The voice alarm may be programmed to state the power level that remains.

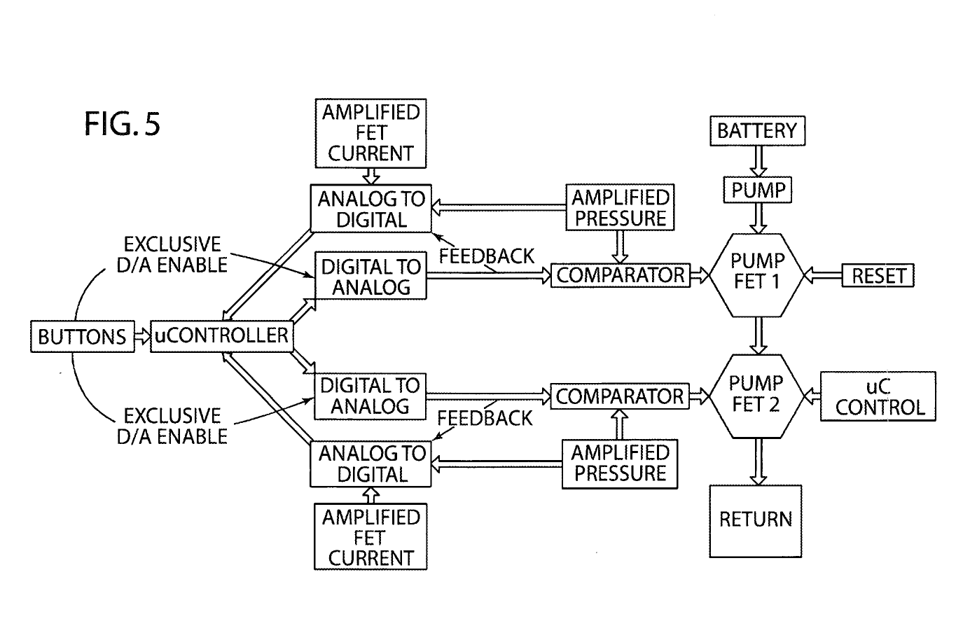

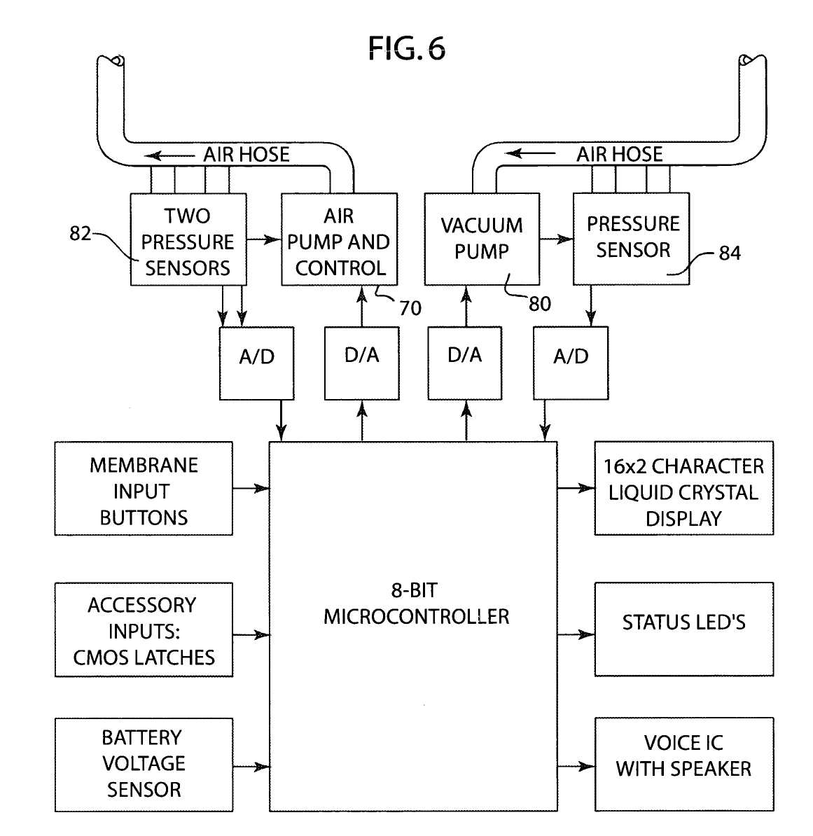

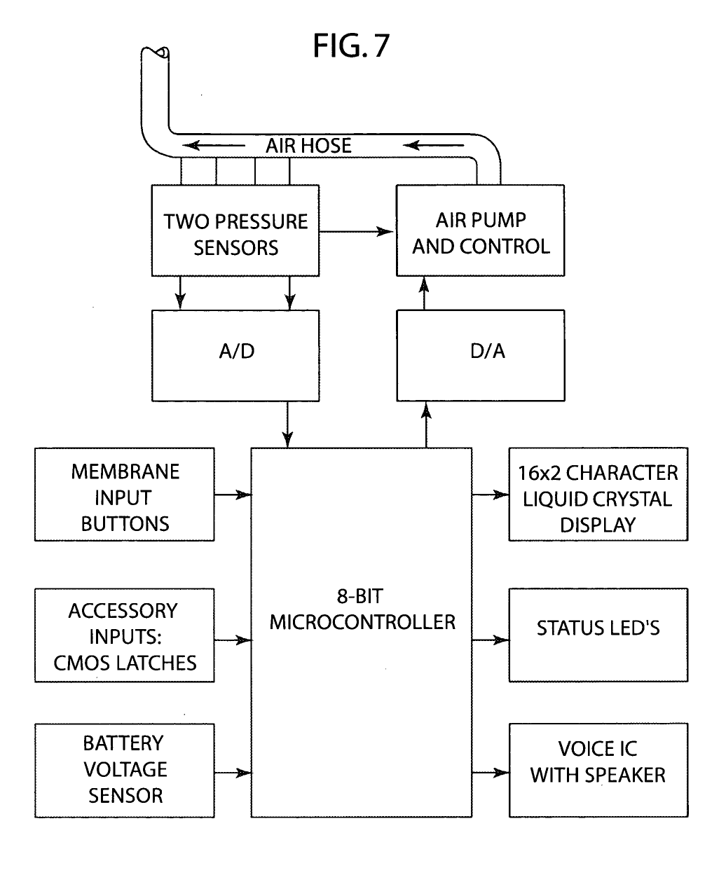

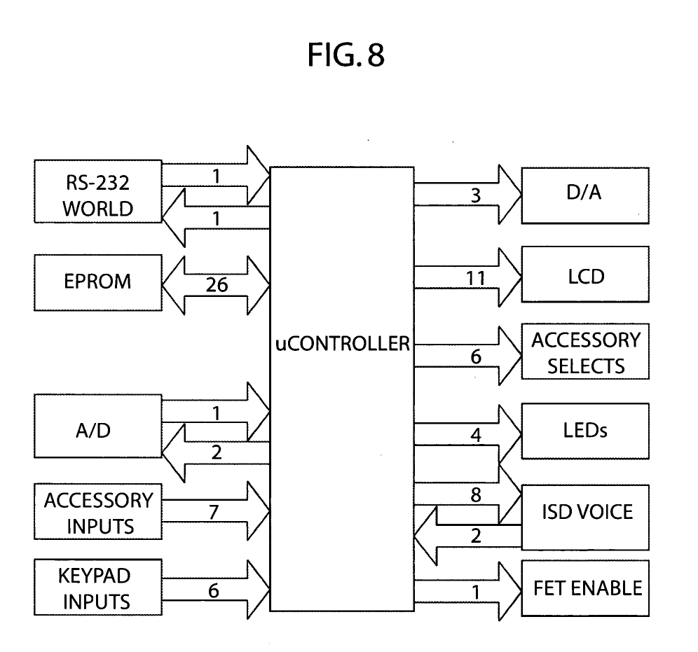

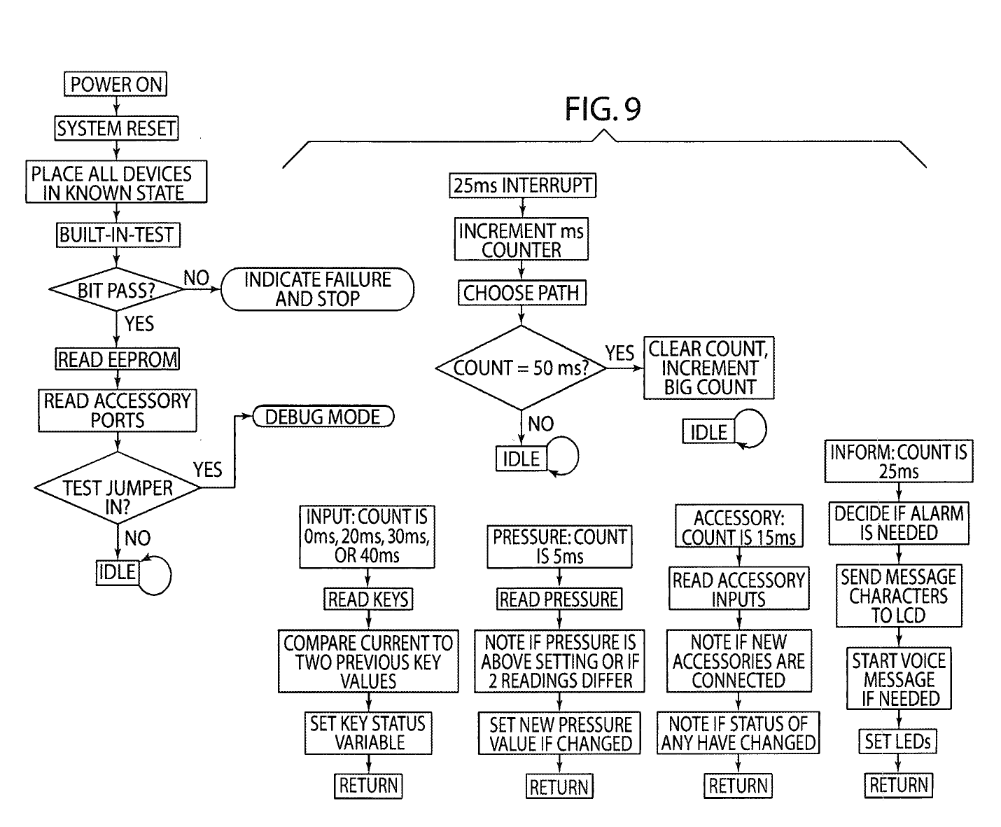

The patient monitor may preferably also be provided with an air pressure pump 70 and may also have a vacuum pump 80 (FIGS. 5-7). Alternatively, a manufacturer or user may prefer to omit the vacuum pump 80 or the pressure pump 70, depending upon their monitoring requirements (FIG. 7).

The pressure pump 70 is useful with infusion bags and may provide a capacity of about 500 mm Hg pressure, for example, by connection of an infusion bag air pressure tube with an air pressure connector of the patient monitor. Dual redundant air pressure sensors 82 are preferably used to monitor selected and used air pressure within about five percent of a predetermined setting. The patient monitor may further be programmed to facilitate a pressure tubing alarm. The pressure tubing alarm may sense a blocked or kinked tube when a set pressure is achieved too quickly, especially in consideration of a specific installed air pump 70. Conversely, an open or leaking pressure tube may be sensed when a set pressure takes too long to achieve. Further, a pressure tubing alarm may be set for when either an infusion bag fluid tube or a pressure tube becomes blocked or kinked after a set pressure is properly achieved.

Similarly, the vacuum pump 80 is useful with suction canisters, a preferred expansible medical suction canister 250 being discussed in greater detail below, by connection of a canister suction tube with a suction connector of the patient monitor. A vacuum sensor 84 preferably monitors the selected and used vacuum level within about five percent of a predetermined setting. The patient monitor may also be programmed to facilitate vacuum tubing alarms. The vacuum tubing alarm may sense a blocked or kinked tube when a set vacuum is achieved too quickly, especially in consideration of a specific installed vacuum pump 80. Conversely, an open or leaking tube may be sensed when a set vacuum takes too long to achieve. Further, a vacuum alarm may be set if either a suction canister tube or a vacuum tube becomes block or kinked after a set pressure is properly achieved.

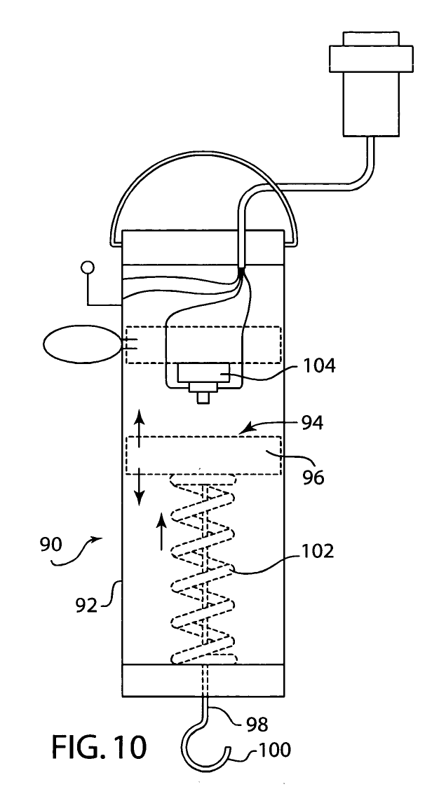

Infusion bag fluid level monitoring may be provided with a mass differential unit 90 such as is shown in FIG. 10. This type of sensor may also be used for any consumable. The fluid bag level sensor 90 may have a generally cylindrical housing 92. The housing 92 may be constructed of any suitable structural material, including metals, ceramics, and plastics, for example. The housing 92 may further be constructed by any appropriate method that is suitable for the material selected. The use of a cylindrical housing 92 may facilitate manufacture by extrusion, for example. Further, the cylindrical shape facilitates cleaning of the sensor.

As shown, the sensor 90 includes a plunger 94 that has a stop 96, a shaft 98, and a connector 100. The fluid sensor 90 will commonly be used by hanging the sensor on an I.V. pole or the like as is commonly known. The plunger 94 is slidibly mounted in the cylinder 92 and slides between extended and retracted positions. A helical scale spring 102 or other scalable bias device urges the plunger 94 toward the retracted position. Further, a micro switch 104 or the like is preferably mounted to close a sensor circuit when the plunger 94 is in the retracted position. The plunger 94 extends when a mass, which may include an infusion fluid bag, is connected with the plunger by connecting the bag with the connector 100, which may be a hook, for example. Conversely, the bias 102 retracts the plunger 94 when the mass diminishes, indicating consumption of the infusion fluid or other consumable.

A trip level of the sensor 90, where the switch 104 is actuated, may be made adjustable. As shown, a position of the switch 104 may be adjustable relative to the spring 102 at 200 ml and 500 ml of infusion fluid, for example. The trip level of the sensor 90 relative to the content level of the infusion fluid is adjustable by relocating the position of the switch 104 along the cylinder 92, as will be understood by one having ordinary skill in the art.

Conversely, the structure of the sensor 90 may be inverted to sense an accumulation of a used fluid, for example, by either having the switch close when the plunger extends away from the switch 104 or by reprogramming the processor to accept an open switch 104 as a signal criteria. Further, the switch 104 may alternatively be placed to contact the plunger stop 96 and close the switch when the plunger is in the extended position. Yet another variation includes configuring the mass differential unit 90 with an integrated signal device as a “stand alone” monitor or with a wireless signal transmitter, including WY-FI technology, for example. One having ordinary skill in the art will understand that these are only a few of many variations that may be used within the concept of the invention.

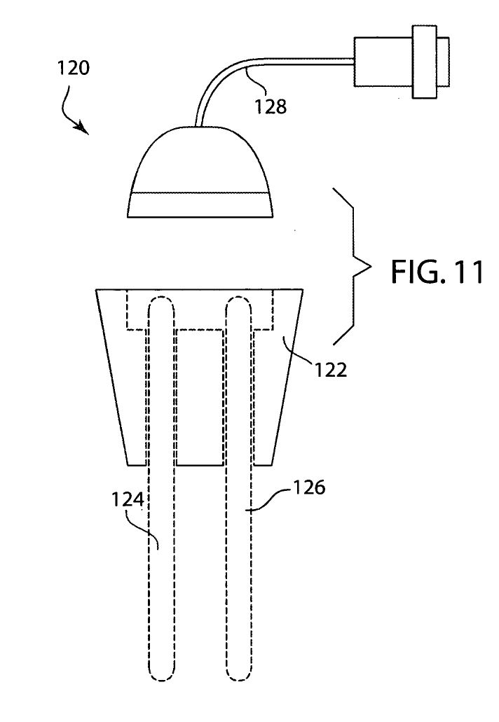

Another fluid accumulation sensor 120 that is useful with suction canisters and the like is shown in FIG. 11. The suction canister level sensor 120 has a plug or stopper 122 that cooperates with an opening in a top or cap of a suction canister. A pair of probes 124 and 126 extend in the same general direction from the stopper 122 and extend into a suction canister when the sensor is mounted for use. As fluid is accumulated in the suction canister, the fluid will make contact with the probes 124 and 126 and make a closed circuit condition that the processor may be programmed to interpret. The sensor 120 is connected with the processor by a cord 128, which may be removable from the stopper 122. Thus, the stopper 122 and probes 124 and 126 may be provided as a disposable part, while the cord 128 may be reusable.

The processor may be programmed to identify a variety of conditions relative to the position of the fluid along the probes 124 and 126. A full condition may be interpreted when the probes 124 and 126 are both first contacted by the fluid. Alternatively, the probes 124 and 126 may extend relatively far into the suction canister and a varying resistance between the probes as the fluid level rises may be interpreted by the processor as progressively greater quantities of fluid in the canister. In one variation, the fluid accumulation sensor 120 may be configured as a “stand alone” monitor with an integrated signal device as one having ordinary skill in the art will understand. In another, alternative variation, the fluid accumulation sensor 120 may incorporate a wireless transmitter.

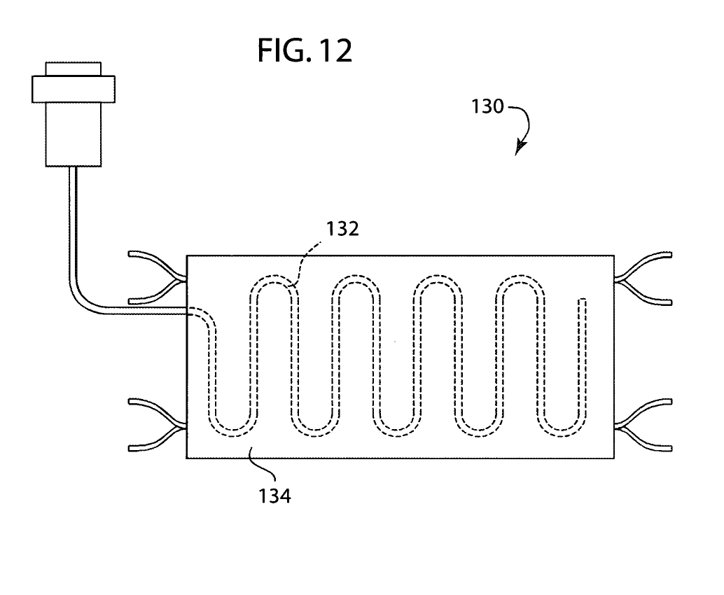

Another useful sensor 130 may include a patient motion sensor. The patient motion sensor 130 shown in FIG. 12 comprises a piezoelectric coaxial cable 132, for example. The cable 132 is threaded through a pad 134 that is placed under a patient. Electrical signals are generated by the piezoelectric cable 132 because of changes in pressure applied to the piezoelectric cable as the patient moves while laying upon the pad 134 and cable. Alternatively, if a more sophisticated motion sensor report is desired, a pad that has a touch sensitive grid may be used. Such a sensor may report specific body position and may be useful in a sleep study, for example.

A preferred medical bed pad 134 may comprise at least two layers and may further be capable of use for easy adjustment of patients within a bed or allows for easy transfer of a patient from one bed to another bed or medical table, for example. Generally, a medical bed pad 134 according to the present invention comprises a padding layer and at least one sheet layer that is adjacent the padding layer. The padding layer is comprised of a pad or mat and provides support for the bed pad of the invention. The sheet layer is a layer adjacent to the padding layer, and provides the surface upon which the patient lies.

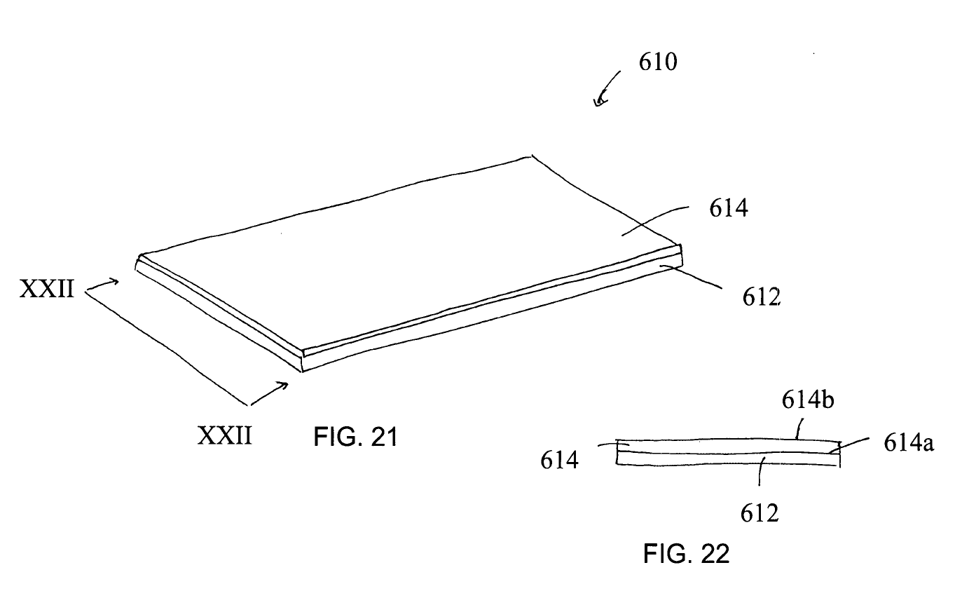

In a simple configuration, a preferred embodiment 134 is shown in FIGS. 21 and 22, a medical bed pad according to the invention is a two layer or laminate construction comprising a padding layer 612 and a sheet layer 614 adjacent to the padding layer. The sheet layer comprises two surfaces, one surface (614a) which is in contact with a surface of the padding layer, and an exposed surface 614b. The exposed surface provides the outer surface of the medical bed pad and is generally the surface upon which the patient lies. Alternatively, in this configuration, a patient could be placed on the exposed surface of the padding layer, i.e., the surface which is not in direct contact with a surface of the sheet layer. However, due to the materials from which the respective layers are made (discussed further herein), it is preferred that the patient lie or be placed upon the sheet layer.

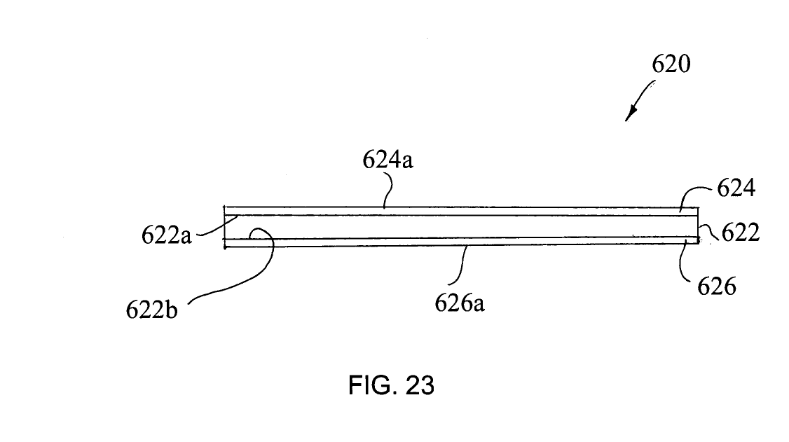

A second embodiment of a medical bed pad according to the present invention is shown in FIG. 23 and may include a three layer laminate or sandwich construction comprising a padding layer 622 having a first surface 622a and a second surface 622b, a first sheet layer 624 adjacent to one of the surfaces of the padding layer, and a second sheet layer 626 adjacent to the other surface of the padding layer. The two sheet layers provide a medical bed pad having two exposed sheet surfaces, e.g. 624a and 626a, upon which the patient may lie. In the medical bed pad depicted in FIG. 23, the sheet layers have the same dimensions as the padding layer. Thus the periphery or sides of the padding layers are exposed. Optionally, the sheet layers may have dimensions such that a portion of the ends and sides of the sheet layers extend beyond the ends and sides of the padding layer. The excess portions of the sheet layers may then be attached to one another, thereby fully enclosing the padding layer.

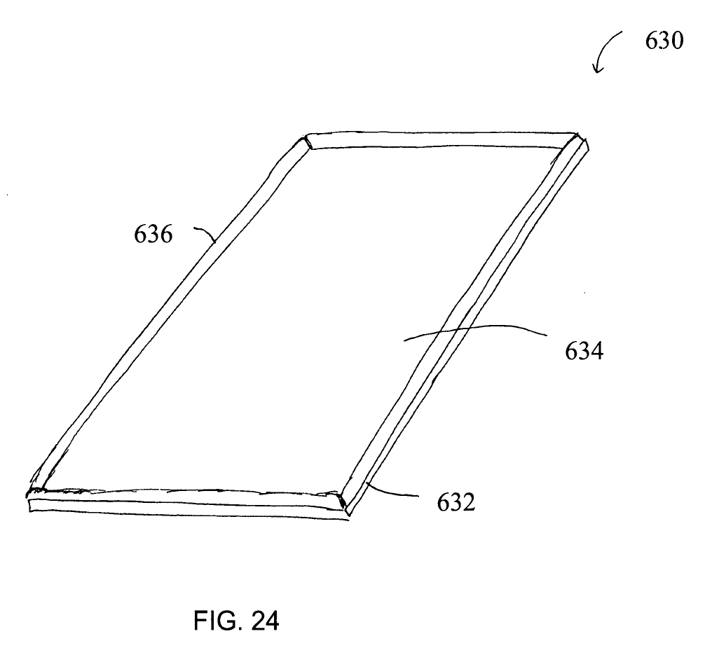

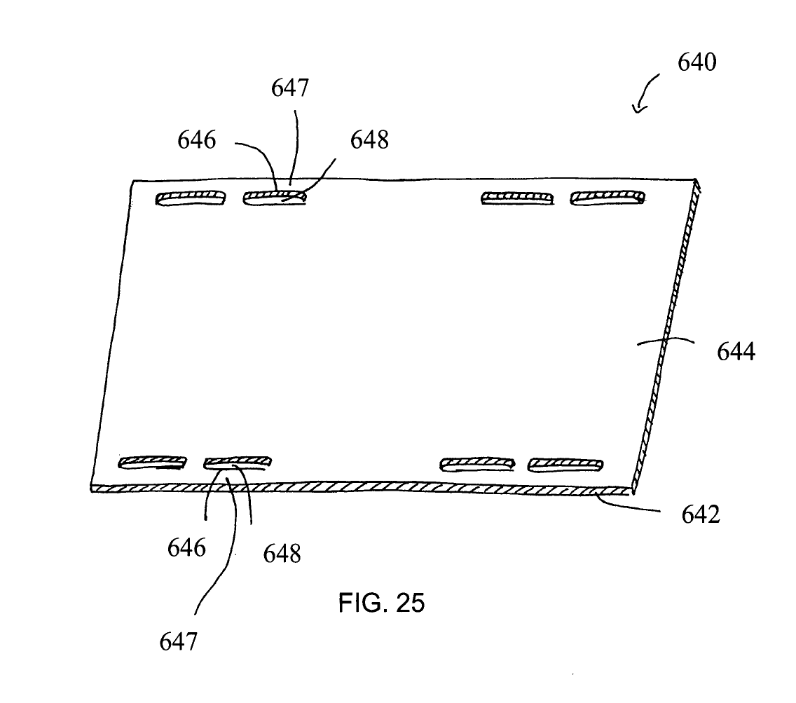

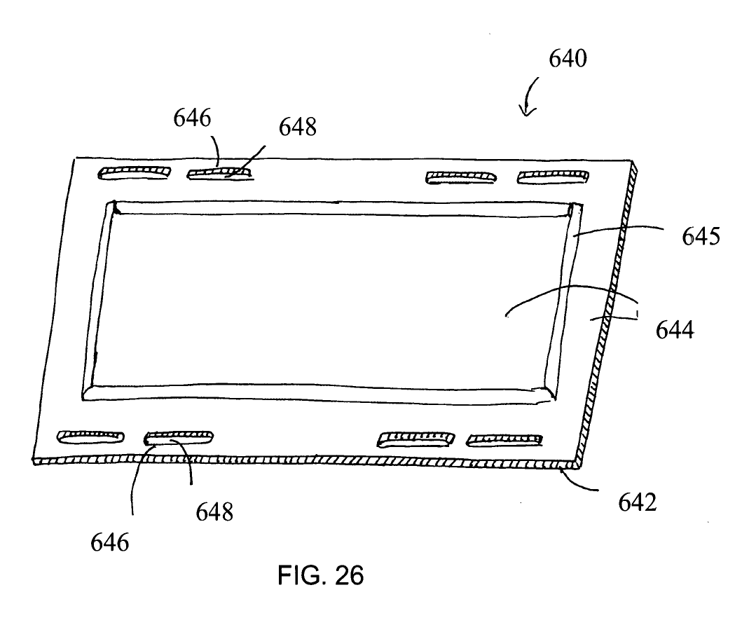

Additionally, a medical bed pad may have a border extending from the top surface of the bed pad, i.e., extending from the exposed surface of the sheet layer. The embodiments depicted in FIGS. 24 and 25 include such a border. In FIG. 24, the medical bed pad 630 comprises a padding layer 632 and a sheet layer 634 adjacent thereto (similar to the medical bed pad depicted in FIGS. 21 and 22). The bed pad 630 further comprises a border 636 adjacent to the top surface of the top layer and extending around the periphery of the sheet layer. FIG. 26 depicts another embodiment that includes a border. In FIG. 26, medical bed pad 640 has a padding layer 642, a sheet layer 644 adjacent to the padding layer, and a border 645 adjacent to and extending from the surface of sheet layer 644. In the embodiment in FIG. 26, the border 645 does not extend around the periphery of medical bed pad 640, as did the border 636 in the embodiment in FIG. 24. Rather, in FIG. 26, the border is contained within the top surface of the medical bed pad 640 such that portions of the sheet layer are exposed on the sides and ends of the medical pad between the border and edges or periphery of the bed pad. A border has a height of about 0.25 inches (6 mm) and a width from about 0.5 inches to about 1.5 inches (13-38 mm). Preferably, the border is a foam material. More preferably, the foam material of the border is adapted to collect and retain liquids within the pad. In place of foam, other liquid absorbent materials may be used. The border may be attached to the sheet layer by any suitable means, including but not limited to the use of adhesive, stitching, sewing, stapling, and thermal bonding techniques. The border may be included to prevent any fluids from running off of the mat onto the bed, floor, or person handling the patient. While FIGS. 24-26 depict the use of a border in conjunction with a two layer medical bed pad construction, the present invention contemplates the use of a border in conjunction with a three layer or sandwich construction. When used with a three layer construction, preferably a border is attached to only one of the sheet layers.

A medical bed pad may also include a plurality of means for gripping, grasping, or taking hold of the medical bed pad to facilitate moving the bed pad (and the patient lying thereon). Any suitable means for gripping or taking hold of the medical bed pad may be included, including but not limited to holes or openings in the medical bed pad and/or handles. Embodiments of medical bed pads that include a means to grip a bed pad are depicted in FIGS. 25-27. The embodiments in FIGS. 25 and 26 show medical bed pad 640 having padding layer 642 and sheet layer 644 adjacent to padding layer 642. Medical bed pad 640 further includes a plurality of holes or openings 648. The openings are longitudinal slots 646 that are formed by portions of the medical bed pad which have been removed. A person may insert their hands or fingers through the openings to grasp the pad. Specifically, the slots essentially create a handle 647 on the surface of the pad in the space between the opening 648 and the edge of the pad. The holes or openings that provide a means to grasp or hold a medical bed pad may have any desired shape or size and are not limited to the longitudinal slots depicted in FIGS. 25 and 26. Additionally, the number of holes or openings may be chosen based on the size of the pad and/or the end users needs.

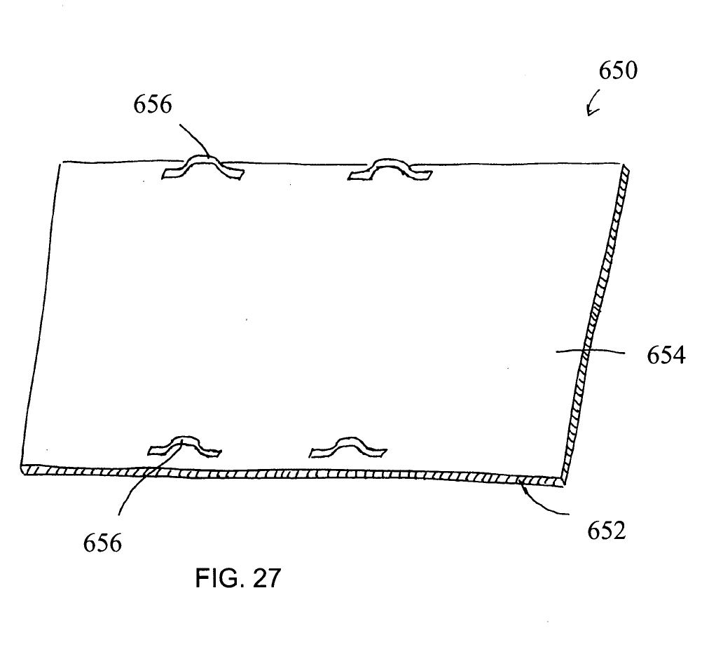

In FIG. 27, medical bed pad 650, has a padding layer 652 and sheet layer 654 adjacent thereto. The pad 650 further includes a plurality of handles 656. As depicted in FIG. 27, the handles are strap handles made from a flexible and/or manipulable material. The straps may be made from any flexible material that is sufficiently strong enough such that the strap will not break during lifting. Non-limiting examples of suitable materials for a strap include nylon or nylon products (e.g., woven nylon materials). In the case of a strap handle, the handle is created by attaching the ends of the material to the medical bed pad, such that a portion of the strap between the ends of the straps (that have been attached to the bed pad) is not attached to the bed pad. The unattached portion of the strap, when manipulated, provides a space between the surface of the bed pad and the underside of the strap through which a person may insert their fingers to take hold of the strap. Retention straps may also be used as the handle. Alternatively, the handles may be made from more rigid materials, including but not limited to plastics, metals or the like. Where a handle is made from a rigid material, it may be desirable to place some type of soft padding material around the handle. The use of such material would provide comfort to personnel handling the pad and also protect patients lying on the pad from the hard, rigid handles. As with the use of holes or openings for the means to grasp the pad, there is no limit with respect to the size, shape, or quantity of handles used on a medical bed pad. Further the location of the plurality means to grasp the bed is not limited in any manner. Preferably the means to grasp the medical bed pad are located to provide for sufficient, uniform support of the patient when the pad is lifted or moved.

The dimensions of a medical bed pad according to the invention may vary according to the needs of the end user. The dimensions of the bed pad generally will conform to the dimensions of beds utilized in a hospital, nursing home, or other medical or patient care environment. Preferably the bed pad will have a length such that the patient=s entire body is capable of making contact with the pad. That is, it is preferred that no part of the patient extend beyond or hang off of the edges of the bed pad. Preferably, the bed pad will have a width of about 36 inches and a length of from about 60 inches to about 72 inches. The size of the pad may also vary depending upon the use. Emergency usage pads will generally be smaller, having a width of about 24 inches and a length of about 60 inches. The foregoing dimensions are merely exemplary and are not intended to limit the scope of the concept in any way. It is contemplated that a medical bed pad may have larger or smaller dimensions as desired and/or needed by an end user.

A medical bed pad preferably has a thickness of from about 0.125 inches (3 mm) to about 1 inch (25 mm), more preferably has a thickness of from about 0.125 inches (3 mm) to about 0.375 inches (10 mm) and most preferably about 0.125 (3 mm) inches to about 0.188 inches (5 mm). It is preferred that the pad be as thin as possible for the patient to lie on. The pad, however, must have a thickness sufficient to support the patient during the transfer or movement of a patient.

It may also be desirable to include a system or sensor, a variation of the fluid accumulation sensor 120 discussed above, for example, to monitor and/or detect when fluids are gathering on the bed pad. A medical bed pad may include, in another embodiment (not shown), one or more sensors disposed between the sheet layer and the padding layer. Additionally, since the sheet layer is preferably fluid repellant and/or impervious to the flow of fluids, sensors may be placed on the top surface of the sheet layer. Sensors may be placed at any location desirable, and are preferably located in regions likely to receive moisture. For example, it is advantageous to utilize sensors in regions of the bed pad that would be expected to receive moisture from the discharge of urine or other electrolytic bodily fluids. Sensors preferably have a minimal thickness such that the flexibility and comfort of the medical bed transfer pad are not compromised. Any suitable moisture detector or sensor may be used as the sensor, including but not limited to the moisture monitoring systems disclosed in U.S. Pat. No. 6,292,102 and/or U.S. Pat. No. 6,580,013, both of which are incorporated herein by reference. Employing a moisture sensor/detector system in a medical bed pad provides several advantages. The use of moisture sensors or detectors provides a means to notify hospital or nursing home personnel when a patient needs assistance. Sensors notify patient care personnel when a patient's bedding needs to be changed or cleaned, when a medical bed pad needs to be cleaned, and/or when a patient needs further care or assistance. Such systems also allow medical or patient care personnel to monitor a patient's condition (for example, whether an incontinent patients condition is improving or not). The use of moisture detectors further allow medical personnel to record a patient's fluid activity. Recording such activity is useful to monitor the patient's condition (such as the time intervals between such activity) and to monitor the time it takes for patient care personnel to respond and provide the appropriate assistance to the patient. Thus, the use of moisture sensors in conjunction with the medical bed pad is advantageous and provides better patient care.

Any suitable material may be used as the padding layer. The material should be soft and pliable enough to provide the patient with a comfortable surface upon which to lie, but rigid enough such that it will adequately support the patient's weight when the pad is used to move the patient. Additionally, it is preferable that the material used in the padding layer is a breathable material. A non-limiting example of a material suitable for use as the padding is a 10 oz needled mat of polyethylene terephthalate (PET) thermobond fiber. Another suitable composition includes a fiber composition comprising from about 10% to about 25% of Rayon, from about 30% to about 40% PET, and from about 30% to about 40% of a PET bi-component. The PET bi-component is a polymer containing PET and a polyolefin. Any polyolefin may be used in the PET bi-component, including but not limited to olefins having 2-30 carbon atoms. Particularly suitable (poly)olefins include polyolefins of ethylene, propylene, butene, pentene, hexene, heptene, octene, nonene, decene, undecene, and dodecene. A particularly preferred polyolefin is polyethylene. Another suitable composition used to form the padding layer is a fiber composition comprising from about 60% to about 80% PET and from about 20% to about 40% Rayon. Additionally, cotton may be added to the fiber compositions in minor amounts to make the pad softer if desired. Cotton may be added in amounts of up to about 25% of the composition. In forming the padding layer, it is desirable to needle or entangle the fibers. Needling the fibers increases the strength of the pad. It is also preferable that the padding layer exhibit antibacterial properties. The padding layer may be treated with an antibacterial component after the pad is formed. Preferably, an antibacterial agent or material is included in the fiber composition. An antibacterial component may be added to the fiber composition in from about 10% to about 15% by weight of the fiber composition. Any suitable antibacterial agent or material known or used in the art may be used in the padding layer.

The sheet layer is preferably made of a fabric material. The fabric material is a breathable sheet that is impervious to liquids. That is, it is preferred that liquids or fluids not be capable of flowing through the sheet layer (and subsequently into the padding layer). Preferably, the fabric is treated with a substance that will make the fabric repellant to fluids such as water, alcohol, blood and the like. Additionally, it is desirable that the fabric is capable of being sterilized by any method known in the art. Because, as discussed further herein, a medical bed pad in accordance with the present development may be used in place of traditional hospital beddings, it is highly desirable that the fabric material provide a suitable surface upon which a patient may lie. That is, it is desirable for the fabric to provide softness and comfort comparable to traditional beddings, with the attendant advantage of being fluid repellant and capable of being sterilized. A suitable material for the outer fabric layer is a non-woven fabric such as DEXTER7 7844 Drape and Gown from Ahlstrom Fiber Composites, Windsor Locks Plant, Two Elm Street, Windsor Locks, Conn. 06096-2335. The sheet layer may have any color desired by the end user. By custom and tradition, hospital beddings typically have a blue color. It may, therefore, be preferred that the material used to form the sheet layer of medical bed pads used in hospital environments has a blue color.

A medical bed pad is formed by providing a padding layer and a sheet layer and attaching the sheet layer to the padding layer. The sheet layer is attached to the padding layer by any suitable means known in the art. A preferred way of attaching the sheet layer to the padding layer is by laminating the sheet layer to the padding layer using an adhesive such as a powdered adhesive or a sheet adhesive. If desirable, the attachment of the sheet layer to the padding layer may be reinforced by further stitching portions of the sheet layer to the padding layer. To reinforce the attachment of the sheet layer to the padding layer, such as by stitching, any portion of the sheet layer may be stitched to the padding layer. For example, the sheet layer may be stitched around the interior of the sheet layer (i.e., along each side at a given distance from the end of the sheet/pad), or the sheet may be stitched to the padding layer along one or multiple locations of either the length and/or width of the pad. The sheet layer may have the same dimensions as the padding layer, such that when the sheet layer is attached to the padding layer the peripheral edges of the padding layer would be exposed. Optionally, the sheet layer may be dimensioned such that the sheet layer has a length or width greater than the length or width of the padding layer. In such configurations, the ends of the sheet layer are pulled over the edges of the padding layer and attached to the underside of the padding layer (by any suitable means, including but not limited to sewing, stitching or laminating) so that the sheet layer essentially forms a seal around the edges of the padding layer. Such a configuration may be desirable to prevent contamination of the padding layer should it come in contact with fluids that escape from the sheet layer. Encompassing the padding layer's edges with portions of the sheet layer is not necessary, however, if the padding layer includes or is treated with an antibacterial agent or material.

Where a medical bed pad includes handles or gripping means, the handles or gripping means may be attached or formed at any appropriate time. Handles, such as for example, strap handles are preferably attached to the medical bed pad after the sheet layer has been attached to the padding layer. Strap handles may be attached to the pad by attaching the ends of the strap to the pad by any suitable means such as sewing, stitching, stapling, bolting, or the like. A portion of the material used for the strap remains separated, i.e., unattached, from the medical bed pad. The unattached portion creates the handle and allows a user to take hold of the strap. In the case of preformed handles made from a solid, rigid material, such as plastic or metal, the handles are attached to the medical bed pad by any suitable means, including but not limited to bolting, screwing, clamping and the like. Removable retention straps are attached to the pad by a clip. The clip may secure the strap to the pad by forming a tight connection over the top of an end of the strap and the underside of the padding layer. Alternatively, the clip may extend through an opening on the strap and through an opening extending through both the sheet layer and the padding layer. The use of a clip allows a handle to be attached to the bed pad when needed to move the patient. Removable handles also allows the handle to be removed when it is not needed, such that the handles do not provide an obstruction to the patient while lying on the pad. Where the gripping means are holes or openings in the pad, such as slots cut along the periphery of the medical bed pad, the gripping means may be formed at any time during the information of the medical bed pad. For example, holes or openings may be formed in the padding layer by cutting and removing a portion of the padding layer to form the holes or openings having a desired shape. Holes or openings may then be formed on the sheet layer corresponding in location, shape and size to the openings in the padding layer. The openings may be formed in the sheet layer either prior to or after the sheet layer is attached to the padding layer. Alternatively, openings may be formed in a medical bed pad after the sheet layer has been attached to the padding layer. In the case of a medical bed pad having openings to provide a means to grip or hold the medical bed pad, the periphery of the openings may further be stitched to reinforce the attachment of the sheet layer to the padding layer and to prevent the sheet layer from fraying or pulling away from the padding layer at those locations.

The bed pad according to the present invention provides for another layer of padding on the bed. The bed pad is placed on top of the mattress of a bed and provides another thin, soft layer for the patient to lay on. The patient is then placed on top of the medical bed pad according to the invention. The bed pad typically just rests on top of the mattress or table. A medical bed pad may also include a means to further secure the bed pad to a mattress or table. For example a bed pad may include an elastic band attached to and extending from either the sheet layer or the padding layer. Preferably an elastic band would be attached to the sheet layer. The band preferably extends around the periphery of the medical bed pad. An elastic band should have a sufficient width such that the band is capable of being pulled or stretched over the periphery of the mattress or table to form a tight connection between the bed pad and the mattress or table. Alternatively, a medical bed pad may include a material layer extending from and around the periphery of the sheet layer. The bottom of the material layer, i.e., the portion of the material layer that is furthest away from the sheet layer, preferably includes a strip of an elastic material similar to that found in traditional bed sheets. The material layer may be pulled over the mattress or table in a manner similar to traditional bed sheets. The use of such means allows a medical bed pad to be secured to a mattress or table to reduce the risk that the pad may slip or slide off the bed or table.

The bed pad, however, also provides for a means of adjusting the patient within the bed, or for moving a patient from one bed to another bed or table. Specifically, the pad is thin enough that it is easily manipulated and handled by medical personnel, but strong enough such that the entire pad may support the patient and allow for the patient to be moved by moving the pad without having to make physical contact with the patient. To change the position of the patient within the bed, medical personnel may grab hold of the pad and slide the pad in the direction necessary to move the patient. With respect to the transfer of a patient from one bed to another, personnel may grab hold of the pad and either physically lift the pad from the first bed and place it on the second bed, or similar to moving the patient=s position, slide the pad off of the first bed and onto the second bed. As previously described herein, the pad may also contain means to grasp or take hold of the pad, including slots, handles, or the like. As previously described herein the gripping means provides a place for persons to grasp or take hold of the pad. Upon grasping the pad via the gripping means, persons may then lift and/or slide the medical bed pad to either change the patient's position, and/or move the patient from one bed to another.

The use of the medical bed pad according to the present invention requires fewer persons to move the patient compared to moving the patient by physically lifting, i.e., handling, the patient's body. Further, because the patient is lying on a uniform surface, the patient's limbs and extremities are not subjected to the jostling and random motion encountered in physically handling the patient. The bed pad according to the invention also has the advantage of being flexible and capable of conforming to the patient's body as compared to hard boards that are sometimes used to move a patient. The flexibility provides additional comfort and allows the patient to lie in a more natural position than if they were on a hard board. Further, the pad preferably contains no additional chemical components.

A medical bed pad in accordance with present development may be used either in place of or in conjunction with traditional hospital beddings. A medical bed pad according to the development may be used in place of traditional beddings. First, a medical bed pad may be placed on a mattress as is commonly found in hospitals, nursing homes, or other patient care facilities. Such mattresses are typically capable of being adjustable so as to allow a portion of the patient's body to be positioned in an incline and/or declined position. The thickness of the medical bed pad imparts flexibility to the bed pad such that the bed pad is capable of conforming to the position of the mattress.

Further, the sheet layer allows a medical bed transfer pad to be used in place of traditional beddings. As previously described herein, the sheet layer is comprised of a fabric material. The fabric material provides a soft surface, comparable to traditional beddings, upon which the patient lies. Thus the sheet layer of the medical bed pad serves the same role as that of a traditional bed sheet. The use of a fluid repellant fabric also makes the medical bed pad superior to traditional beddings. As previously described herein, the fabric material of the sheet preferably exhibits fluid repellant properties. The fluid repellant properties prevent absorption into the sheet layer and the padding layer of the bed pad. Thus, fluids are retained on the surface of the medical bed pad such that the medical bed pad provides a liquid holding pad. By retaining fluids on the surface of the pad, risks involved from fluids leaking to the floor are reduced. Additionally, risks to hospital personnel from making contact with the fluids during movement of the patient are also reduced. The pad also has the advantage of being cleaned more easily than traditional bedding. Specifically, the fluid repellant properties of the fabric comprising the sheet layer and the consequent fluid retaining feature of the pad makes the pad capable of being cleaned more easily then traditional beddings that readily absorb fluids. The pad may be wiped down, washed off and then sterilized. The absorbent properties of traditional beddings may make cleaning traditional bed sheets more difficult or impossible. It may take repeated attempts in between uses of traditional beddings to completely clean the bedding, or it may not be possible to sufficiently clean the bedding for reuse. Consequently, great expense may be involved in either cleaning and/or replacing traditional hospital beddings. Thus, a medical bed pad according to this development, which is easily cleaned and reused, may provide significant reductions in costs associated with cleaning and/or replacing traditional beddings. A medical bed pad is disposable, and easily replaced. Preferably, a bed pad will be used only once before it is discarded. The fluid repellant properties of the sheet layer, as previously described, however, also allows a medical bed pad to be easily cleaned, sterilized and reused. The ability to be reused may be desirable for at-home patient care or where it is financially desirable to reuse the pads. The fluid repellant and easy cleaning properties of the fabric of the sheet layer may also reduce the frequency with which the bedding must be changed and/or replaced. This reduces the cost of replacing the bedding, but also reduces the number of times that a patient needs to be moved.

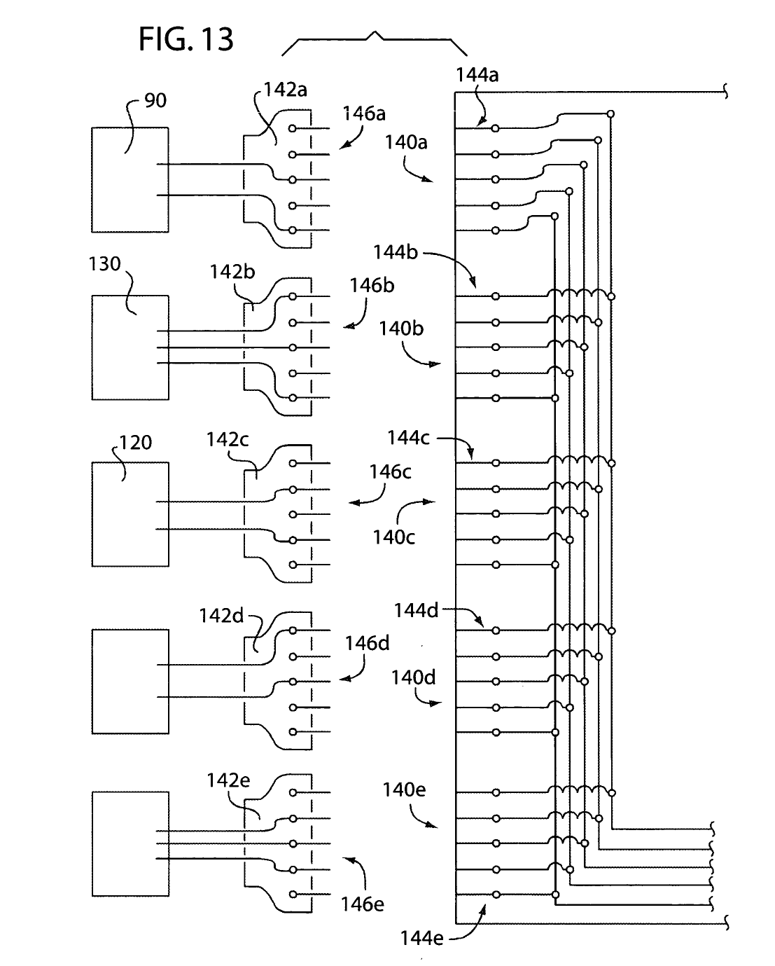

The use of a wide variety of sensors is provided in the patient monitor by using keyed input sockets 140a-140e and plugs 142a-142e (FIG. 13). The cooperating input sockets 140a-140e and plugs 142a-142e are not sensor specific in that a different socket and plug combination are designated for each sensor. Rather, a socket 140a-140e and plug 142a-142e combination that has a plethora of connectors or contacts 144a-144e and 146a-146e, respectively, is used and a pattern or set of the contacts is identified with a particular sensor, resulting in a smart plug connection of the sensor with the processor. An exemplary schematic of five plugs 142a-142e and five sockets 140a-140e is shown in FIG. 13. Each plug 142a-142e and socket 140a-140e arbitrarily has five contacts 144a-144e and 146a-146e, respectively, as shown in FIG. 13. Significantly, any of the plugs 142a-142e can mate with any of the sockets 140a-140e. The sockets 140a-140e are conventionally connected in parallel, so the sockets are electrically identical. The distinction comes in how the socket contacts 144a-144e are connected with the processor and how the processor is programmed to interpret various signals from the contacts. One having ordinary skill in the art will understand that the processor may be programmed to discern and report as desired virtually any combination of contact input. Thus, a programming of the processor may dictate a plug wiring of the sensors or vice versa, a predetermined plug wiring of the sensors may dictate a programming of the processor.

As shown, the processor may be programmed to accommodate a plug 142a that has contacts 146a wired for a sensor 90 such as an infusion bag level empty sensor signal, while also accommodating a plug 142c that has contacts 146c wired for a sensor 120 such as a suction canister level full signal, for example. A plug 142b that has contacts 146b wired for a sensor 130 such as a piezoelectric motion sensor input may also be accommodated, for example. Other desired sensors may further be accommodated by differentiated wiring of contacts 146d and 146e of plugs 142d and 142e. Thus, the wiring of the sensor with the plug 142a-142e, and more particularly with the plug contacts 146a-146e and so with the processor through the socket contacts 144a-144e differentiates or identifies the particular sensor. A significant feature of this cooperating socket 140a-140e and plug 142a-142e arrangement is that the sensors and the processor may be configured so a preselected sensor can be plugged into any socket 140a-140e without a user wasting time or being distracted or otherwise being confused or making a mistake with regard to identifying the correct socket before plugging in a sensor.

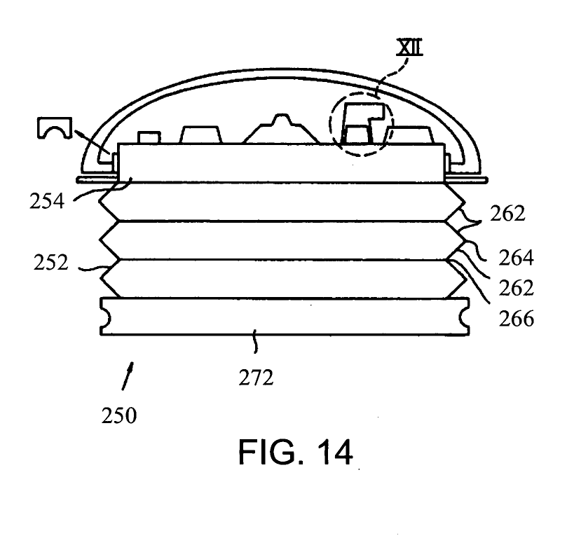

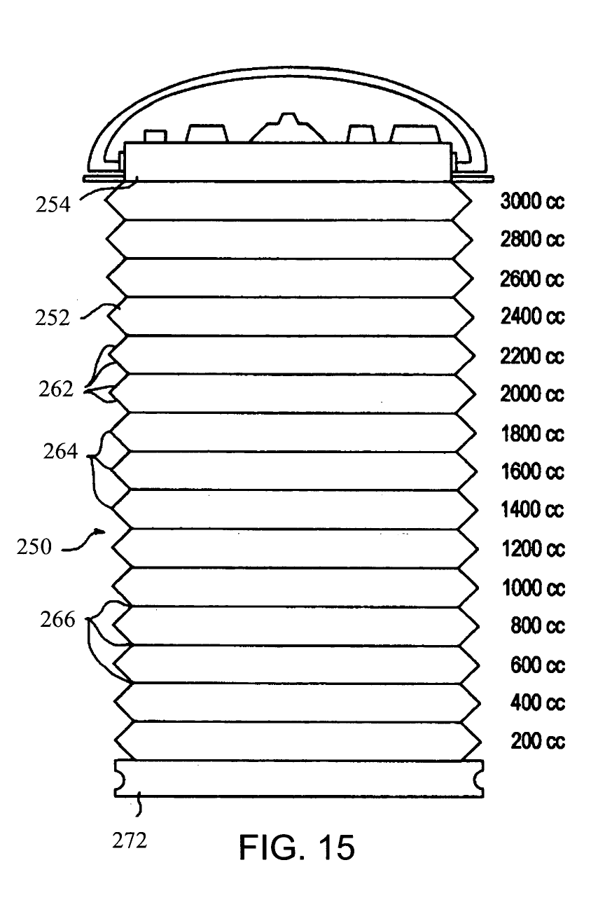

A preferred embodiment of an expansible medical suction canister 250 according to the invention is generally shown in the drawing FIGS. 14 and 15 and discussed below. The expansible canister 250 may have a telescoping body 252 that defines a volume adjustable inner chamber. The canister 250 may also have a lid 254. The canister 250 may expand and compress between open and closed or expanded and compressed positions. While the body 252 may be provided with various telescoping or extensible configurations, as one having ordinary skill in the art will understand from this disclosure, the body 252 most preferably has a generally cylindrical side wall that is defined by an accordion or bellows configuration. Further, the body 252 is most preferably a one piece member that is constructed of a durable moldable material, including and not limited to, polyvinyl plastic.

More particularly, the side wall has a series of fan fold rings 262 that are hingedly interconnected at peaks 264 and valleys 266 by what is commonly known as a living hinge. If taken individually, each ring 262 defines a conic frustum. The side wall may be considered an array of alternating conic frustums that are hinged together. A bottom 272 may be integrally formed with the body 252 at one of two opposing ends, while a generally cylindrical ring is formed at the other end of the body and adapted to couple with the lid 254. The lid 254 and ring may be non-releasably snap-fit together or may be releasably screw-fit together, for example, as commonly known to one having ordinary skill in the art. Although, the canister may also be constructed without a lid per se. When provided as a disposable item of medical equipment, a user may prefer a non-releasable coupling of the lid 254 with the body 252. Clearly, the lid feature of the canister is quite variable and adaptable to a user's requirements.

Because the interior chamber is adjustable or expandable, provision must be made to lock or hold the chamber volume at a preselected size when a suction is drawn on the canister in use. One having ordinary skill in the art will understand that a volume adjustable canister will have an inherent tendency to collapse when a suction is applied to the interior chamber. By variation of the thickness of the rings and living hinges, and of the relative dimensions of the inner and outer diameters of the rings, compression and expansion force characteristics of the canister are varied. That is, the amount of suction that is required to collapse the canister may be adjusted and preset in manufacture of the canister, according to the specifications of the sidewall elements. An ability to sustain a suction of about thirty pounds per square inch (30 psi) is sufficient in most surgical use settings, for example.

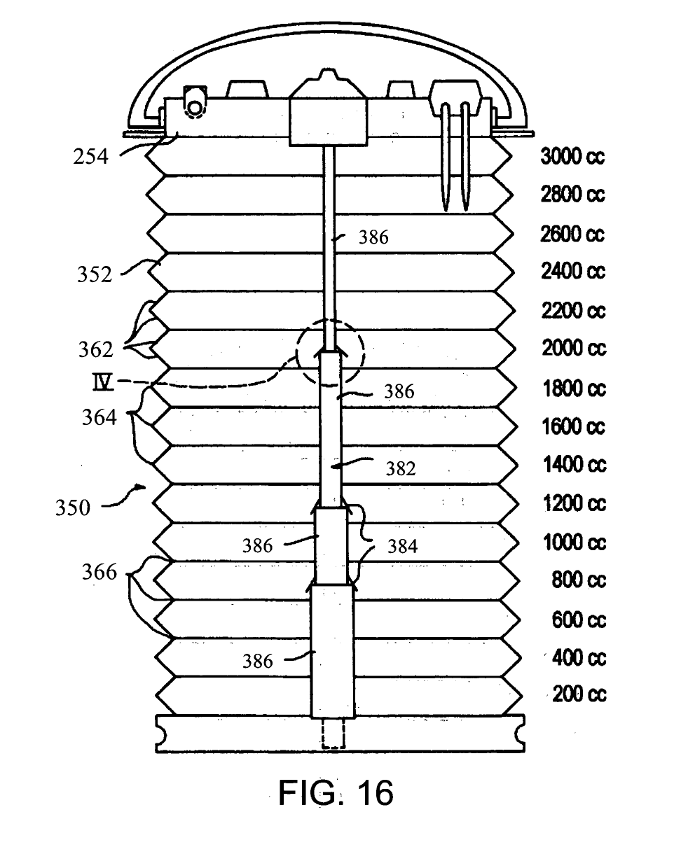

In an alternative embodiment 350 of an expansible suction canister (FIGS. 16-20), the body 352 is constructed as a bellows with rings 362 and having hinges at peaks 364 and valleys 366, that are sized for relative ease of expansion and compression of the canister 350, a bellows being old and well known. Thus, a new combination with a locking support 382 is provided to hold the canister 350 at a preselected volume expansion. One having ordinary skill in the art will know that the side wall of the expansible canister may have various configurations or constructions, including concentric telescoping tubes, for example, when an alternative support, including and not limited specifically to support 382, resists compressive forces applied when a canister 350 is under suction.

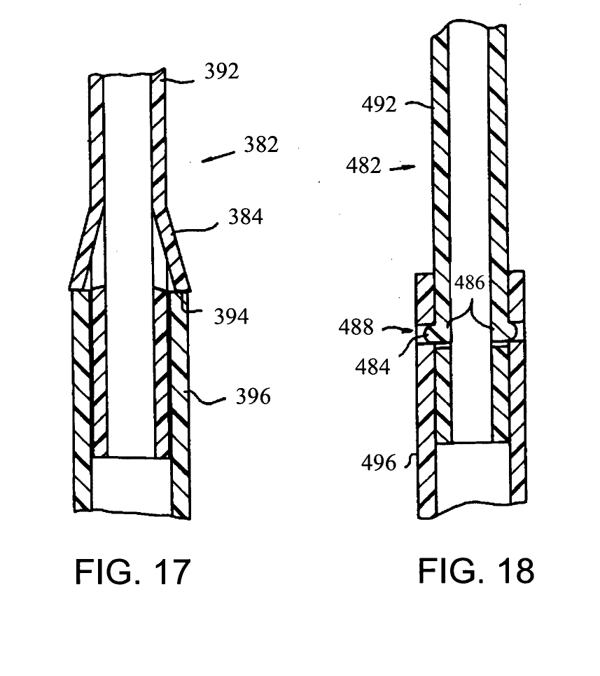

The support 382 may be a telescoping member, which has a series of coaxial sliding elements or tubes 386 as is commonly known. Frangible legs 384 (FIGS. 16 and 17) may be provided to hold the elements 186 at predetermined extended positions. The legs 384 hold the support 382 in a preselected extended position during use and may be forced to collapse when a user presses the canister down for disposal.

The legs 384 may be conveniently formed by punching, or the like, a portion of a side wall of an element 386. By constructing the element 186 of a length of plastic or metal tubing, for example, the legs 386 may be made to bias outward. Thus, the legs of an element 392 will spring out and abut an end 394 of an adjoining element 396 (FIG. 17), the end 394 defining a stop surface. Further, by selection of the material and thickness of a side wall of the element 392, a force required to collapse the legs 384 and the canister 350 may be predetermined as will be understood by one having ordinary skill in the art.

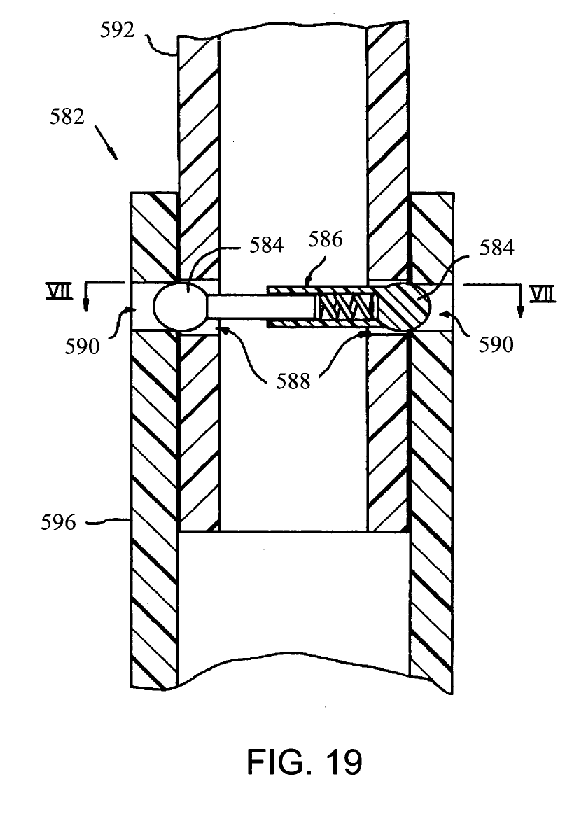

In a first alternative 482 of the support 382, the legs 384 may be replaced with detents 484 on flexible tabs 486, or the like (FIG. 18). The detents 484 on one element 492 mate with cooperating detent receptacles, namely, apertures 488 of an adjoining element 496. In yet a second alternative 582 of the support 382, the legs 384 may be replaced with detents 584 on a spring rod 586, or the like (FIG. 19). The detents 584 slide freely through guides in the form of apertures 588 through one element 592 and mate in abutting engagement with cooperating detent receptacles or apertures 590 of an adjoining element 596. One having ordinary skill in the art will understand that any of the detents 484, the detents 584, the apertures 488 and the apertures 590 may be constructed with any of various cross-sectional geometries, or surface contours. Thus, a given detent geometry paired with a given aperture geometry will provide a particular strength of the locking support, either 482 or 582, relative to a force that may be applied to the canister and effect a compressing of the canister, including a suction that is drawn on the canister, for example. That is to say that a force that is required to compress the canister, or the resistance to that force, may be adjusted or pre-set by selection of the detent and the aperture geometries, and more particularly, specific pairings of the detent and the aperture geometries. It is a given, then, that the supports 482 and 582 may be constructed with a predetermined resistance to a canister compression force, and that the resistance may range from precluding compression of an open canister to virtually not resisting compression of an open canister.

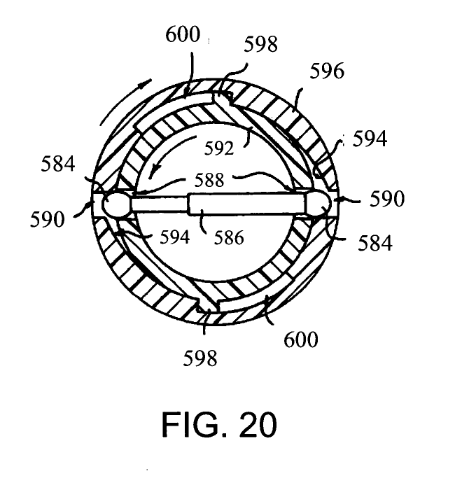

A release mechanism may further be provided with regard to either of the supports 482 and 582. While the release mechanism may be applied equally well to either of the supports 482 and 582, as will be understood from this disclosure by one having ordinary skill in the art, the release mechanism will be explained with reference to locking support 582 (FIG. 20). A rotary cam surface 594 may be formed in alignment with each aperture 590. Then by rotation of the elements 592 and 596 as shown by the arrows, the detents 584 are pressed inward and disengage the apertures 590. A rotation limit may also be employed by way of a tab 598 and slot 600.

One having ordinary skill in the art and those who practice the invention will understand that various modifications and improvements may be made without departing from the spirit of the disclosed inventive concept. One will also understand that various relational terms, including left, right, front, back, top, and bottom, for example, are used in the detailed description of the invention and in the claims only to convey relative positioning of various elements of the claimed invention. The scope of protection afforded is to be determined by the claims and by the breadth of interpretation allowed by law.

I claim:

1. A patient monitoring device in combination with a fluid reservoir, comprising:
a processing device;
a display operatively connected with the processing device;
a selector operatively connected with the processing device;
a housing;
at least one air pump mounted in the housing and operatively connected with the processing device, the air pump being adapted to provide at least one of air pressure and air suction;
a fluid reservoir operatively connected with the air pump, the air pump providing suction to the reservoir, a suction canister that is adapted to collect fluid when the air pump applies a suction to the suction canister, the fluid reservoir being connected with the air pump so that fluid is precluded from drawing into the air pump;
at least two sockets, each socket being substantially identical with a number of socket connectors, the sockets being electrically connected in parallel with one another, the socket connectors being operatively connected with the processing device;
a first sensor, the first sensor being adapted to generate a signal according to a predefined criteria, the first sensor having a first sensor plug that corresponds to the at least two sockets whereby the first sensor plug removably couples with any of the at least two sockets, the first sensor plug having a set of first plug connectors that cooperates with the socket connectors, the first sensor being connected with fewer than all of the socket connectors when the first sensor plug is coupled with either of the at least two sockets and defining a first subset of the socket connectors, the first subset of the socket connectors being those socket connectors that connect with the first sensor when the first sensor plug is coupled with one of the at least two sockets; and
a second sensor, the second sensor being adapted to generate a signal according to a predefined criteria, the second sensor having a second sensor plug that corresponds to the at least two sockets whereby the second sensor plug removably couples with any of the at least two sockets, the second sensor plug having a set of second plug connectors that cooperates with the socket connectors, the second sensor being connected with fewer than all of the socket connectors when the second sensor plug is coupled with either of the at least two sockets and defining a second subset of the socket connectors, the second subset of the socket connectors being those socket connectors that connect with the second sensor when the second sensor plug is coupled with one of the at least two sockets, the second subset of the socket connectors also being a different subset from the first subset of the socket connectors; and
an expansible medical suction canister comprising:
a body, the body having a top and an opposing bottom; the body defining an expansible interior chamber between the top and the bottom, the chamber being expandable to an open position and being compressible from the open position to a closed position; the body including one of a bellows and of at least first and second concentric tubes telescopingly engaged in liquid tight engagement; and
a support connected between the top and the bottom, the support being adapted to releasably lock the chamber in the open position.

2. The combination of claim 1, wherein the support is a telescoping member, the support having a first element and a second element, the first element including a detent, and the detent engaging the second element.

3. The combination of claim 2 wherein the detent is a leg, wherein the second element has a stop surface, and the leg has an end that abuts the stop surface.

4. The combination of claim 3 wherein the leg is frangible.

5. The combination of claim 2 wherein the detent has a surface that engages the second element.

6. The combination of claim 5 wherein the detent is biased toward engagement with the second element.

7. The combination of claim 6 wherein the second element has a cooperating detent receptacle.

8. The combination of claim 2 wherein the second element has a cooperating detent receptacle.

9. A patient monitoring device in combination with a fluid reservoir, comprising:
a processing device;
a display operatively connected with the processing device;
a selector operatively connected with the processing device;
a housing;
at least one air pump mounted in the housing and operatively connected with the processing device, the air pump being adapted to provide at least one of air pressure and air suction;
a fluid reservoir operatively connected with the air pump, the air pump providing suction to the reservoir, a suction canister that is adapted to collect fluid when the air pump applies a suction to the suction canister, the fluid reservoir being connected with the air pump so that fluid is precluded from drawing into the air pump;
at least two sockets, each socket being substantially identical with a number of socket connectors, the sockets being electrically connected in parallel with one another, the socket connectors being operatively connected with the processing device;
a first sensor, the first sensor being adapted to generate a signal according to a predefined criteria, the first sensor having a first sensor plug that corresponds to the at least two sockets whereby the first sensor plug removably couples with any of the at least two sockets, the first sensor plug having a set of first plug connectors that cooperates with the socket connectors, the first sensor being connected with fewer than all of the socket connectors when the first sensor plug is coupled with either of the at least two sockets and defining a first subset of the socket connectors, the first subset of the socket connectors being those socket connectors that connect with the first sensor when the first sensor plug is coupled with one of the at least two sockets; and
a second sensor, the second sensor being adapted to generate a signal according to a predefined criteria, the second sensor having a second sensor plug that corresponds to the at least two sockets whereby the second sensor plug removably couples with any of the at least two sockets, the second sensor plug having a set of second plug connectors that cooperates with the socket connectors, the second sensor being connected with fewer than all of the socket connectors when the second sensor plug is coupled with either of the at least two sockets and defining a second subset of the socket connectors, the second subset of the socket connectors being those socket connectors that connect with the second sensor when the second sensor plug is coupled with one of the at least two sockets, the second subset of the socket connectors also being a different subset from the first subset of the socket connectors; and
an expansible medical suction canister comprising:
an expansible chamber, the chamber being expandable to an open position and being compressible from the open position to a closed position, the chamber having a first end and an opposing second end, the first and the second ends being spaced relatively farther apart in the open position than in the closed position; and
a support, the support being connected between the opposing ends, the support moving with the opposing ends between the open and closed positions, the support having a releasable locked condition in the open position.

10. The expansible medical suction canister of claim 9 wherein the support has a first element and a second element, wherein the first and the second elements move between open and closed positions with the canister, wherein the first element has a stop surface, wherein the second element includes a leg with an end that abuts the stop surface in the open position and compression of the canister to the closed position is resisted.

11. A patient monitoring device in combination with a fluid reservoir, comprising:
a processing device;
a display operatively connected with the processing device;
a selector operatively connected with the processing device;
a housing;
at least one air pump mounted in the housing and operatively connected with the processing device, the air pump being adapted to provide at least one of air pressure and air suction;
a fluid reservoir operatively connected with the air pump, the air pump providing suction to the reservoir, a suction canister that is adapted to collect fluid when the air pump applies a suction to the suction canister, the fluid reservoir being connected with the air pump so that fluid is precluded from drawing into the air pump;
at least two sockets, each socket being substantially identical with a number of socket connectors, the sockets being electrically connected in parallel with one another, the socket connectors being operatively connected with the processing device;
a first sensor, the first sensor being adapted to generate a signal according to a predefined criteria, the first sensor having a first sensor plug that corresponds to the at least two sockets whereby the first sensor plug removably couples with any of the at least two sockets, the first sensor plug having a set of first plug connectors that cooperates with the socket connectors, the first sensor being connected with fewer than all of the socket connectors when the first sensor plug is coupled with either of the at least two sockets and defining a first subset of the socket connectors, the first subset of the socket connectors being those socket connectors that connect with the first sensor when the first sensor plug is coupled with one of the at least two sockets; and
a second sensor, the second sensor being adapted to generate a signal according to a predefined criteria, the second sensor having a second sensor plug that corresponds to the at least two sockets whereby the second sensor plug removably couples with any of the at least two sockets, the second sensor plug having a set of second plug connectors that cooperates with the socket connectors, the second sensor being connected with fewer than all of the socket connectors when the second sensor plug is coupled with either of the at least two sockets and defining a second subset of the socket connectors, the second subset of the socket connectors being those socket connectors that connect with the second sensor when the second sensor plug is coupled with one of the at least two sockets, the second subset of the socket connectors also being a different subset from the first subset of the socket connectors; and
an expansible medical suction canister comprising:
an expansible chamber, the chamber being expandable to an open position and being compressible from the open position to a closed position, the chamber having a first end and an opposing second end, the first and the second ends being spaced relatively farther apart in the open position than in the closed position; and
a support, the support being connected between the opposing ends, the support moving with the opposing ends between the open and closed positions, the support having a locked condition in the open position, the support being a telescoping member, the support having a first element and a second element, the first element including a detent, and the detent engaging the second element.

12. The expansible medical suction canister of claim 11 wherein the detent is biased toward engagement with the second element.
